(19) United States
(12) Patent Application Publication
(10) Pub. No.: US 2020/0061109 A1
(43) Pub. Date: Feb. 27, 2020

(54) ZINC CHLORIDE HYDROXIDE HAVING EXCELLENT ZINC ION SUSTAINED-RELEASABILITY AND PRODUCTION METHOD THEREFOR

(71) Applicant: JFE MINERAL COMPANY, LTD., Tokyo (JP)

(72) Inventors: Yoshimi NAKATA, Tokyo (JP); Yuko ECHIZENYA, Tokyo (JP); Etsurou UDAGAWA, Tokyo (JP); Osamu YAMAMOTO, Yamagata (JP)

(21) Appl. No.: 16/467,852

(22) PCT Filed: Dec. 8, 2017
(86) PCT No.: PCT/JP2017/044220

§ 371 Date: Jun. 7, 2019

(30) Foreign Application Priority Data
Dec. 9, 2016 (JP) 2016-239802

(51) Int. Cl. A61K 33/30; A61L 15/18; A61L 15/44; C01G 9/04; A61P 17/02

(52) U.S. Cl. CPC A61K 33/30; A61L 15/18; A61P 17/02; C01G 9/04; A61L 15/44

(57) ABSTRACT

Provided are: zinc chloride hydroxide that is useful as a pharmaceutical ingredient, has excellent zinc ion sustained-releasbility when used as a pharmaceutical agent, and exhibits excellent stability as a pharmaceutical ingredient; and a production method therefor. Disclosed is zinc chloride hydroxide that can be used as a pharmaceutical ingredient, that has excellent zinc ion sustained-releasability, that includes at least one member selected from the group consisting of zinc chloride, zinc hydroxide, and zinc oxide.

### TECHNICAL FIELD

The present invention relates to a zinc chloride hydroxide having excellent zinc ion sustained-release properties that can be used as an active ingredient of pharmaceuticals and a method for manufacturing the same.

### BACKGROUND ART

Patent Literature 1 describes in paragraph [0013] that, regarding the zinc supplements, “it is found that administration of relatively high amounts of well absorbed forms of zinc prior to, or concurrently with therapeutic administration of botulinus toxin will enable responsiveness to the toxin in individuals who were previously poorly responsive, and apparently enhance the functional potency of botulinus toxins in other individuals as well”. Paragraph [0045] of Patent Literature 1 describes, as examples of such “zinc supplements”, inorganic zinc or organic zinc or a combination thereof for oral administration, and zinc chloride (ZnCl₂), basic zinc chloride (Zn₅Cl₂(OH)₈), zinc oxide (ZnO), and zinc sulfate (ZnSO₄) are listed.

However, use of basic zinc chloride such as simonkolleite as an active ingredient of pharmaceuticals has not been studied.

Zinc is believed to be a substance that supplies Zn²⁺ ions to a wound site when used as a therapeutic agent for a skin wound or skin roughness. A technique in which Zn²⁺ ions can be appropriately supplied to a wound site, from the perspective of an active ingredient of pharmaceuticals, the substance can be supplied in the form of zinc chloride hydroxide having excellent stability, and in addition, the substance can be provided through a simple and convenient manufacturing method, is desired.

### CITATION LIST

### Patent Literature

Patent Literature 1: JP 2012-531421 A

### SUMMARY OF INVENTION

### Technical Problem

The invention aims to provide a zinc chloride hydroxide useful as an active ingredient of pharmaceuticals, having excellent zinc ion sustained-release properties and having excellent stability as an active ingredient of pharmaceuticals, when used as pharmaceuticals, and a method for manufacturing the same.

### Solution to Problems

The active ingredient of pharmaceuticals mentioned later is characterized as an inorganic compound composition containing a zinc chloride hydroxide that can be used in the pharmaceuticals described below. Generally, inorganic compound compositions which contain zinc are of a plate-like shape and they are known to be used in cosmetics (applications to the skin) utilizing their concealing properties. However, to be used as an active ingredient of pharmaceuticals, a plate-like shape may cause a risk of cytotoxicity, and thus, it is preferably an atypical shape, an ellipsoid shape, a substantially spherical shape without sharp parts with a particle size of from 50 to 0.1 μm, and more preferably a particle size of from 10 to 1 μm. More preferably, depending on the application of the pharmaceutical product, adjustments such as particle size, surface area, particle size distribution, may be made so that the sustained-release of zinc ions from the pharmaceuticals can be appropriately controlled.

The present description discloses, as pharmaceuticals, a therapeutic agent for a skin wound or skin roughness, a medical device having a therapeutic agent and a covering material, and a set in which a therapeutic agent and a covering material are combined. The invention describes a zinc chloride hydroxide having excellent zinc ion sustained-release properties that can be used as an active ingredient of these pharmaceuticals, and a method for manufacturing the same.

(Zinc Chloride Hydroxide having Excellent Zinc Ion Sustained-Release Properties that can be used as an Active Ingredient of Pharmaceuticals)
(1) A zinc chloride hydroxide having an excellent zinc ion sustained-release property, which can be used as an active ingredient of a pharmaceutical, the zinc chloride hydroxide comprising at least one selected from the group consisting of zinc chloride, zinc hydroxide, and zinc oxide.
(2) The zinc chloride hydroxide having an excellent zinc ion sustained-release property according to (1), which can be used as an active ingredient of a pharmaceutical, wherein the zinc chloride comprises at least one selected from the group consisting of zinc chloride, zinc chloride hydroxide, and zinc chloride hydroxide hydrate.
(3) A zinc chloride hydroxide having an excellent zinc ion sustained-release property that can be used as an active ingredient of a pharmaceutical, wherein an amount of dissolved Zn²⁺ ions is from 0.25 to 100 μg/m² per unit surface area, and pH is not less than 7.0 and less than 8.3, after a dissolution test by stirring method performed on a simonkolleite-containing zinc chloride hydroxide hydrate;

where, in the dissolution test, a BET specific surface area of the simonkolleite-containing zinc chloride hydroxide hydrate is from 10 to 150 m²/g, a mass ratio of the simonkolleite-containing zinc chloride hydroxide hydrate to saline is 1:50, and a time for stirring at 37° C. using a rotor at 500 rpm is 3 hours.

(4) The zinc chloride hydroxide having an excellent zinc ion sustained-release property according to (3), which can be used as an active ingredient of a pharmaceutical, wherein a structure of Zn₅Cl₂(OH)₈·nH₂O is dominant in an XRD diffraction pattern of the simonkolleite-containing zinc chloride hydroxide hydrate, where a axis is from 6.3 to 6.345 and c axis is from 23.4 to 23.7.
(5) The zinc chloride hydroxide having an excellent zinc ion sustained-release property that can be used as an active ingredient of a pharmaceutical according to (3) or (4), wherein the simonkolleite-containing zinc chloride hydroxide hydrate is represented by Formula (1) below, and a molar ratio of Zn and Cl is Zn/Cl=2.0−4.0,

Zn₄₋₆Cl₁₋₃(OH)₇₋₈·nH₂O (1)

where n is 0 to 6.

(6) The zinc chloride hydroxide having an excellent zinc ion sustained-release property according to (5), which can be used as an active ingredient of a pharmaceutical, wherein the pharmaceutical is a therapeutic agent for a skin wound or skin roughness, and when the therapeutic agent is dissolved in saline and used, a ratio of the therapeutic agent to saline is from 0.1 g/L to 100 g/L, and the therapeutic agent is represented by Formula (1) above, and when n=0 (anhydrous), a zinc concentration relative to a total amount of the therapeutic agent is from 45 mass % to 75 mass % as metal zinc, and a zinc concentration in the saline aqueous solution of the therapeutic agent is from 0.045 g/L to 75 g/L.
(7) The zinc chloride hydroxide having an excellent zinc ion sustained-release property according to any one of (1) to (6), which can be used as an active ingredient of a pharmaceutical, wherein the pharmaceutical is a therapeutic agent for a skin wound or skin roughness which reaches up to dermis via epidermis, or for pressure ulcers which reach up to peritoneum via epidermis and dermis.
(8) The zinc chloride hydroxide having an excellent zinc ion sustained-release property according to (6) or (7), which can be used as an active ingredient of a pharmaceutical, wherein the therapeutic agent is in the form of powder, lotion, solution, cream, ointment, spray, or gel, and is applied or sprayed to a part of a skin wound or skin roughness to be utilized.
(9) The zinc chloride hydroxide having an excellent zinc ion sustained-release property according to any one of (6) to (8), which can be used as an active ingredient of a pharmaceutical, wherein the pharmaceutical is a medical device comprising a wound covering material for retaining the therapeutic agent and the skin wound or skin roughness in a closed environment.
(10) The zinc chloride hydroxide having an excellent zinc ion sustained-release property according to (9), which can be used as an active ingredient of a pharmaceutical, wherein the wound covering material is at least one selected from the group consisting of polyurethane film dressing material, hydrocolloid dressing material, polyurethane foam dressing material, alginate covering material, hydrogel dressing material, hydropolymers, cellulose film, chitin wound covering material, and silk film.
(11) The zinc chloride hydroxide having an excellent zinc ion sustained-release property according to any one of (6) to (10), which can be used as an active ingredient of a pharmaceutical, wherein the therapeutic agent for a skin wound or skin roughness is present by being applied to, contained in, or adhered to the wound covering material.
(12) The zinc chloride hydroxide having an excellent zinc ion sustained-release property according to any one of (9) to (11), which can be used as an active ingredient of a pharmaceutical, wherein the wound covering material is made to carry the therapeutic agent when manufactured, or the therapeutic agent is fixed through spray, immersion, or application to an application side surface of the wound covering material that has been processed into a sheet form, whereby the therapeutic agent and the wound covering material are used as a medical device.
(13) The zinc chloride hydroxide having an excellent zinc ion sustained-release property according to any one of (1) to (8), which can be used as an active ingredient of a pharmaceutical, wherein the pharmaceutical is a combined set of the therapeutic agent for a skin wound or skin roughness containing the zinc chloride hydroxide having an excellent zinc ion sustained-release property, and the wound covering material for retaining a skin wound or skin roughness in a closed environment.
(Methods for Manufacturing a Zinc Chloride Hydroxide having Excellent Zinc Ion Sustained-Release Properties that can be used as an Active Ingredient of Pharmaceuticals)

For the ultimate use as an active ingredient of pharmaceuticals, the manufacturing method described below comprises: a step of crushing for adjusting into preferably an atypical shape, an ellipsoid shape, or a substantially spherical shape without sharp parts with a particle size of from 50 to 0.1 μm, and more preferably a particle size of from 10 to 1 μm, or a step of adjusting the particle size using a ball mill or the like (including granulation).

(14) A method of manufacturing the zinc chloride hydroxide according to any one of (1) to (13), comprising maintaining an almost constant pH by acid or alkali in the presence of aqueous solution including chloride ions, continuously supplying zinc ions from an aqueous zinc solution, and precipitating zinc chloride hydroxide having a main component of simonkolleite. In the present description, “maintaining an almost constant pH” is not particularly limited and means maintaining preferably at ±1 of a predetermined pH and more preferably at ±0.3 of a predetermined pH. The “main component of simonkolleite” means that zinc chloride hydroxide contains simonkolleite as the most dominant component. Zinc chloride hydroxide may also be referred to as basic zinc hydroxide in the present description.
(15) The method of manufacturing the active ingredient of a zinc chloride hydroxide pharmaceutical according to (14), wherein the pH is not less than 6.0 and less than 7.5, and a temperature is lower than 40° C.
(16) The method of manufacturing the zinc chloride hydroxide according to (14) or (15), wherein the zinc ion donor is at least one selected from the group consisting of zinc chloride and zinc nitrate.
(17) The method of manufacturing the zinc chloride hydroxide according to any one of (14) to (16), wherein the chloride ion donor is at least one selected from the group consisting of ammonium chloride and sodium chloride.

### Advantageous Effects of Invention

The invention is a zinc chloride hydroxide which is useful as an active ingredient of pharmaceuticals and has an excellent stability.

### BRIEF DESCRIPTION OF DRAWINGS

FIG. 1 is a chart of XRD (X-ray diffraction method) of simonkolleite obtained by the method for manufacturing a Preparation Example of the invention. pH is the pH when synthesized.

FIG. 2 shows photographs showing the observation results of a healing site (sample) of a wound site and of a control after one week in Pharmaceutical Example 1.

FIG. 3 shows photographs showing the observation results of the healing site (sample) and of the control after two weeks in Pharmaceutical Example 1.

FIG. 4 is a graph showing the results of Table 2.

FIG. 5 shows micrographs showing histological assessment of a wound created part after one week of treatment using Pharmaceutical Example 1.

FIG. 6 shows micrographs showing histological assessments of a wound created part 2 and a wound created part 3 after two weeks of treatment in Pharmaceutical Example 1. Part 1, part 4, and part 5 other than the wound created parts are shown together.

FIG. 7 is a graph showing the relationship between pH of simonkolleite at synthesis and pH after a dissolution test.

FIG. 8 is a graph showing the relationship between pH of simonkolleite at synthesis and the amount of dissolved Zn²⁺ ions after the dissolution test.

### DESCRIPTION OF EMBODIMENTS

### 1. Summary of Zinc Chloride Hydroxide of the Invention

(1) The zinc chloride hydroxide of the invention is a zinc chloride hydroxide including at least one selected from the group consisting of zinc chloride, zinc hydroxide, and zinc oxide, which has excellent zinc ion sustained-release properties, and can be used as an active ingredient of a pharmaceutical therapeutic agent for a skin wound or skin roughness as below-mentioned.

Zinc chloride hydroxide is a chloride salt of zinc and is preferably represented by the chemical formula Zn₄₋₆Cl₁₋₃(OH)₇₋₈·nH₂O . . . Formula (1), where n is 1 to 6.

In the industrial field, it is known as a generally representative chemical formula: simonkolleite Zn₅(OH)₈Cl₂, and this substance is known to be a dense corrosion product, have an excellent corrosion inhibitive effect, and improve corrosion resistance of a plating layer by promoting the generation of simonkolleite.

Zinc chloride hydroxide of the invention includes at least one selected from the group consisting of zinc chloride, zinc hydroxide, and zinc oxide, and zinc chloride contains at least one selected from the group consisting of zinc chloride, zinc chloride hydroxide, and zinc chloride hydroxide hydrate.

The zinc chloride hydroxide of the invention may be natural or commercially available zinc chloride, zinc hydroxide or zinc oxide, or may be synthesized, or may be a mixture of them. Zinc oxide is described as a pharmaceutical in the Japan Pharmacopoeia.

The zinc chloride hydroxide of the invention may be obtained using a precipitate produced by an alkali precipitation method from an aqueous zinc salt solution. Preferably, in the precipitate generation reaction described below, a precipitate, obtained by the reaction of Zn²⁺ ions, Cl⁻ ions, and OH⁻ ions in a reaction field where the pH is controlled preferably not less than 6.0 and less than 7.5, is used as the zinc chloride hydroxide of the invention. More preferably, it is a zinc chloride hydroxide containing simonkolleite that is a zinc chloride hydroxide hydrate represented by Formula (1) below:

Zn₄₋₆Cl₁₋₃(OH)₇₋₈·nH₂O (1)

where n is 0 to 6.

The zinc chloride hydroxide of the invention has an amount of dissolved Zn²⁺ ions of from 0.25 to 100 μg/m², and pH of not less than 7.0 and less than 8.3, and more preferably an amount of dissolved Zn²⁺ ions of from 10 to 100 μg/m² and pH of not less than 7.0 and less than 8.3 after a dissolution test by the stirring method explained in the Pharmaceutical Examples.

### (Dissolution Test by Stirring Method)

The amount of dissolved Zn²⁺ ions is measured in the present description as follows: The surface areas of samples, which are produced with varying pH values at production by the same process as that for Preparation Example 2 of Examples to be described later, are measured beforehand by the BET method (BET specific surface area analyzer: High Precision, Multi-Analyte Gas Adsorption Analyzer, available from Quantachrome Instruments Japan G.K.). For each sample, the Zn²⁺ ion concentration after stirring in saline is measured by an ICP emission spectrometer (ICPE-9000, available from Shimadzu Corporation) to thereby obtain the amount of dissolved Zn²⁺ ions, and the amount of dissolved Zn²⁺ ions is divided by the surface area previously measured. The mass ratio of each sample to saline is 1:50 and the amount of Zn²⁺ ions dissolved in the saline is measured after stirring at 37° C. for 3 hours at 500 rpm using a rotor.

(2) The active ingredient of pharmaceuticals may be used as a pharmaceutical alone, or it may be used together with a suitable carrier.

(2-1) The pharmaceuticals can be used as a medical device for treating skin wound or skin roughness as well as a wound covering material that retains the zinc chloride hydroxide and the skin wound or skin roughness in a closed environment. The zinc chloride hydroxide is preferably coated, impregnated, or adhered to a wound covering material that retains the therapeutic agent for a skin wound or skin roughness in a closed environment.

(2-2) In another aspect, the pharmaceuticals may be used as a medical set for treating a skin wound or skin roughness, which is a combination of a therapeutic agent for a skin wound or skin roughness containing an zinc chloride hydroxide that can be used as an active ingredient of pharmaceuticals, with the above-mentioned wound covering material.

### 2. Method for Manufacturing Zinc Chloride Hydroxide of the Invention

The chlorine source used in the alkali precipitation method is preferably an aqueous solution of at least one of NaCl or NH₄Cl (ammonium chloride), and more preferably an aqueous NH₄Cl solution. NaOH is added dropwise as a mineralization material so that the pH is maintained preferably at not less than pH 6.0 and less than 7.5, and an acidic aqueous solution of zinc salt such as zinc chloride is added dropwise to form a precipitate, and stirred for 10 to 30 hours to obtain a precipitate. After solid-liquid separation by suction filtration or centrifugation, the precipitate is washed with pure water or distilled water, and dried under vacuum to obtain a dry powder containing simonkolleite. The particle size of the obtained zinc chloride hydroxide hydrate containing simonkolleite is not limited, and when used as an active ingredient of pharmaceuticals such as therapeutic drugs, the particle size can be set to an appropriate particle size by a known method. The chlorine source includes aqueous solution of NaCl or the like, preferably NH₄Cl; the zinc source includes zinc sulfate, zinc chloride, zinc acetate, zinc nitrate and the like, and is preferably selected from zinc chloride and zinc nitrate, and the use of NH₃ or NaOH aqueous solution as the mineralization material is included. The chlorine source aqueous solution and the zinc source aqueous solution are preferably reacted at a concentration ratio (molar ratio) of chlorine to zinc of approximately 2:5, and the concentration of the zinc source aqueous solution is preferably in a range of from 0.1 to 1 M. The reaction is preferably performed at 40° C. or lower, and more preferably at 25° C. The obtained zinc chloride hydroxide hydrate having Simonkolleite as a main component is a mixture of a reaction product obtained by the above-mentioned precipitate generation reaction of an aqueous zinc salt solution and an aqueous alkali solution, raw materials as unreacted products, by-products, and impurities contaminated from raw materials.

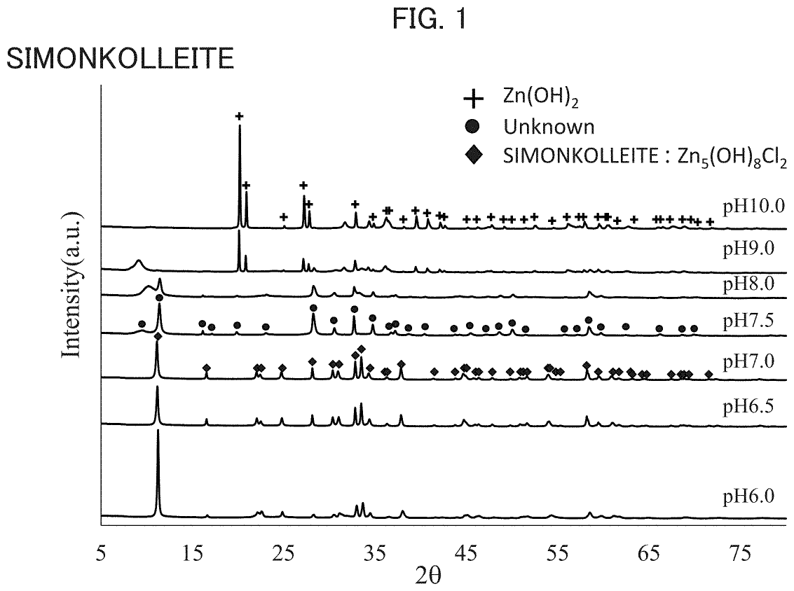

FIG. 1 shows an XRD chart of simonkolleite manufactured with varying pH conditions according to the method of manufacturing a Preparation Example of the invention. As for the XRD device, D8 ADVANCE available from Bruker Corporation is used.

As shown in the cases of pH 6.0 to 7.0 at synthesis in the chart in FIG. 1, at the XRD diffraction peaks of the zinc chloride hydroxide hydrate containing simonkolleite, the structure of Zn₅Cl₂(OH)₈·nH₂O being simonkolleite is dominant, where a axis is preferably from 6.3 to 6.345 and c axis is preferably from 23.4 to 23.7. Within this range of the XRD diffraction peaks, the crystal exhibits a good effect as a therapeutic agent for a skin wound or skin roughness. At pH 7.5 or greater, the unknown peaks are dominant. The manufacturing conditions under which simonkolleite can be obtained as a main component will vary depending on the type and concentration of the chloride source and zinc source used, and an optimal condition can be found by varying the pH conditions for manufacturing. Here, the main component refers to the most abundant component in the mixture, and it is preferably not less than 60 mass %, more preferably not less than 80 mass %, and further preferably not less than 95 mass %.

To date, a large number of studies have been conducted by applying zinc compounds such as ZnSO₄, ZnCl₂, and ZnO to wound sites created in experimental animals to assess wound healing effects. These compounds are substances that supply Zn²⁺ ions to the wound site. However, it has been reported that there is an optimal concentration of Zn²⁺ ions for wound healing effects owing to Zn²⁺ ions. At a concentration of no more than 500 μmol/L, no toxicity is shown against fibroblasts; however, the presence of high level of zinc ions (not less than 15 mmol/L) is known to increase inflammatory cell infiltration of skin and to significantly delay re-epithelialization.

In addition, pH of a body fluid having saline as a main component is approximately 7.4 to 7.5, and when the zinc chloride hydroxide of the invention having simonkolleite as a main component is dissolved in saline, variation of pH that corresponds to generation and dissolution of Zn²⁺ ions is expected. Furthermore, by deciding on the following 2 states, i.e., the pH environment for supplying Zn²⁺ ions and/or Cl⁻ ions and the supply of OH ions for efficiently activating the matrix metalloprotease (MMPs) enzymes that decompose substrate proteins via OH of water molecules, the treatment is further promoted, and tissues that interfere with the treatment, such as scab, would not be formed, the scar remaining is prevented, and thus, the patient's QOL (Quality of Life) can be expected to improve.

In the wound healing process, it is known that active proliferation and migration of cells occurs. When cells migrate into or between tissues, the existing extracellular matrix has to be locally disrupted. A new extracellular matrix is formed simultaneously to reconstruct the tissue at the wound site. Various proteolytic enzymes are involved in these processes.

The active balance of matrix metalloproteases (MMPs) and tissue inhibitory metalloproteases (TIMPs) is responsible for both normal and pathological events, such as wound healing, tissue repair, angiogenesis, infiltration, tumor formation, and metastasis.

MMP is an enzyme that decomposes extracellular collagen and is synthesized by cells that are present between connective tissues. MMP has a zinc ion in the center and there is a Zn²⁺ ion binding site at the active site. Epidermal regeneration is achieved by the epidermis cells migrating from wound edges and skin appendages (hair root, sweat glands, etc.). Thus, binding of Zn²⁺ ions from the zinc compound with MMPs causes destruction of the extracellular matrix and promotes epidermal cell migration.

TIMP is an enzyme that is produced by fibroblasts, endothelial cells, and the like, and has an inhibitory effect against MMP. MMPs and TIMPs form a complex in a ratio of 1:1, and thus the collagen decomposition of MMPs is inhibited. This mechanism can facilitate fiber formation at the wound site by inhibiting specific cleavage of the helix site of the type I, II and III collagens caused by MMPs, and thus, can increase the amount of collagen in the regenerated tissue.

As stated above, it is believed that the zinc chloride hydroxide of the invention can promote cell migration by appropriately supplying zinc ions and/or chloride ions to the wound site, and can increase collagen accumulation and promote wound healing. Furthermore, by focusing on a pH environment for supplying zinc ions and/or chloride ions, the treatment is further promoted, and tissues that interfere with the treatment, such as scab, would not be formed, the scar remaining is prevented, and thus, the patient's QOL (Quality of Life) can be expected to improve.

The pharmaceutical therapeutic agent for a skin wound or skin roughness using the inorganic composition of the invention is not limited but can treat skin wounds or skin roughness. Here, the skin wound or skin roughness is a skin wound or skin roughness which is a loss in epidermis and dermis (of full thickness skin) reaching up to dermis via epidermis, or pressure ulcers, skin wound, or skin roughness which reaches up to peritoneum via epidermis and dermis.

### 3. Other Applications

(1) Pharmaceuticals for use with Wound Covering Materials

The zinc chloride hydroxide having excellent zinc ion sustained-release properties of the invention that can be used as an active ingredient of pharmaceuticals is effective, as pharmaceuticals, for the healing of a skin wound or skin roughness which reaches up to dermis via epidermis, and of pressure ulcers which reaches up to peritoneum via epidermis and dermis. It is effective for the healing of skin wound or skin roughness which is a loss of full thickness skin reaching up to dermis via epidermis.

The pharmaceutical using the invention can be a therapeutic medical device for a skin wound or skin roughness, which is applied to a skin wound or skin roughness and retains the skin wound or skin roughness in a closed environment when used together with a wound covering material. Here, “applied to a skin wound or skin roughness” means that it may be applied directly to the skin wound or skin roughness, or may be applied to the skin around the skin wound or skin roughness. A wound covering material is a medical device that retains the skin wound or skin roughness in a closed environment.

Healing of skin wounds or skin roughness may involve healing in a dry environment and in a moist environment. When healing in a dry environment, no wound covering material may be used in some cases, but generally the wound or skin roughness is covered with a breathable wound covering material for protection. Gauze, bandages, and breathable film-like wound covering materials may be used.

The therapeutic agent for a skin wound or skin roughness can be brought in a closed environment with wound covering materials and be also retained in a suitable moist environment, thereby being therapeutically effective. When the zinc chloride hydroxide of the invention which is an inorganic material is combined with the wound covering material which is an organic material, a large synergistic effect can be obtained by hybridizing the organic and inorganic materials.

When the active ingredient of the invention is used as a pharmaceutical, saline or the like is used as a solvent to make a liquid drug for use in the form of, for example, lotion, solution, cream, ointment, or spray. Furthermore, the active ingredient can also be used in a dosage form of powder body such as powder or of ointment when exudate is present in the wound and the wound is moist. If necessary, it can be made into a thickening state or gel agent (sometimes referred to as a gel) which is an intermediate state of the above. The therapeutic agent for a skin wound or skin roughness using the zinc chloride hydroxide of the invention is therapeutically effective in a moist environment as the therapeutic agent can be brought in a closed environment with the wound covering material and its dosage form can be adjusted into a liquid or powder so as to maintain the individual wound in a suitable moist state. Regardless of whether the dosage form is liquid or powder, when the pharmaceutical therapeutic agent is used with the wound covering material, the tissues, such as scab, that interfere with the treatment would not be formed, and the scar remaining is prevented so that a high QOL (Quality of life) of a patient is obtained.

### <Medical Device Comprising Therapeutic Agent and Wound Covering Material>

In another aspect, the therapeutic agent of the invention may be carried in the covering material in the manufacture of the wound covering material so as to be used in a medical device. This may be a medical device in which the therapeutic agent of the invention is applied to, contained in, or adhered to the wound covering material and the wound covering material retains the skin wound or the skin roughness in a closed environment. The therapeutic agent is contained and deposited when the wound covering material is synthesized.

Alternatively, the therapeutic agent may be fixed by spraying, immersing, or applying the therapeutic agent to the application side surface of the wound covering material that has been processed into a sheet form, and then used as a medical device.

### <Wound Covering Material>

Examples of the wound healing materials can include, polyurethane film dressing material, a hydrocolloid dressing material, polyurethane foam dressing material, alginate covering material, hydrogel dressing material, hydropolymers, chitin wound covering material, and silk film, but are not limited thereto.

For details of the wound covering materials that are used with the pharmaceutical of the invention, reference can be to the description in paragraphs [0029] to [0037] of an international application, PCT/JP2016/067404 filed by the present applicant concerning the case where used along with the zinc chloride hydroxide of the invention.

For example, a chitin wound covering material is described below as an example not described in the above international application.

The chitin would covering material is obtained by removing calcium or proteins which become an allergen from a shell of a crustacean and purifying the resultant to obtain amino polysaccharides, which are formed into a sheet form. This has a high bioaffinity, and analgesic and hemostatic effects can be expected. While the material has an excellent water absorption and allows the retention of moist environments, secondary dressing materials are needed. The chitin processed into a cotton form is thick, and when a large amount of exudate is present, it is replaced daily, and when the amount is reduced, the time till replacement is extended. A gauze coated with the sponge-like processed chitin is also useful.

The therapeutic agents of the invention can be pharmaceuticals sets with these wound covering materials. The form of use (treatment technique) as a pharmaceutical at this time is as follows.

The active ingredient of the invention may be a therapeutic agent for a skin wound or skin roughness as a pharmaceutical, and may include a pharmaceutically acceptable carrier, if necessary. Examples of the carrier include organic powders, inorganic powders, organic solvents and inorganic solvents, more specifically, corn starch, cereal flour, talc, water, saline, alcohols, polyhydric alcohols, or mixtures thereof. To have a form of the pharmaceutical, a thickening agent or the like may be added, and the ingredient is processed into a gel form or a paste form having the improved handleability.

<Carriers used in Therapeutic Agent>

Examples of the carrier include hydrocarbons such as alpha-olefin oligomers, paraffin waxes, ceresin, microcrystalline wax, animal and vegetable oils such as persic oil, olive oil, beef fat, and mink oil, synthetic esters such as cetyl octanoate, isopropyl myristate, and cetyl palmitate, natural animal and vegetable waxes such as jojoba oil, carnauba wax, candelilla wax, Japan wax, and beeswax, silicone oils and derivatives thereof such as sorbitan stearate, polyoxyethylene glyceryl tristearate, polyoxyethylene lauryl ether, decaglyceryl trioleate, sucrose monolaurate ester, dimethylpolysiloxane, and methylphenyl polysiloxane, and the like.

Fluororesins such as perfluoropolyether, alcohols such as ethanol, 1,3-butylene glycol, propylene glycol, and diglycerin; carrageenan, xanthan gum, sodium carboxymethylcellulose, collagen, elastin, silk, cellulose, lactoferrin and other proteins and hydrolysates thereof, powders of anhydrous silicic acid, nylon powder, polyalkyl acrylate, alumina, and iron oxide may be used.

In addition, ultraviolet absorbers, vitamins, ureas, dried-seawater products, anti-inflammatory agents, amino acids and derivatives thereof, lecithin, colorants, perfumes, preservatives, and the like, oils such as egg yolk oil, macadamia nut oil, cottonseed oil, avocado oil, coconut oil, palm oil, palm kernel oil, corn oil, peanut oil, beef fat, and carnauba wax can be used.

Still others include beeswax, liquid paraffin, lanolin, squalane, stearic acid, laurate esters, myristic acid esters, isostearyl alcohol, purified water, electrolyzed water, ethyl alcohol and the like. That is, in general, those commonly blended in cosmetic products and quasi drugs can be used as the carrier in the therapeutic agent for a skin wound or skin roughness of the invention. The carrier may not be used.

For example, other components that may be added to the therapeutic agent for a skin wound or skin roughness of the invention are selected depending on those to be actually added in cosmetics or quasi drug. Although it cannot be strictly distinguished, humectants include glycerin, sorbitol, polyethylene glycol, pyrrolidone carboxylic acid and salts thereof, collagen, 1,3-butylene glycol, hyaluronic acid and salts thereof, chondroitin sulfate and salts thereof, xanthan gum, and the like.

Antioxidants include ascorbic acid, α-tocopherol, dibutylhydroxytoluene, parahydroxyanisole, and the like. Surfactants include sodium stearyl sulfate, diethanolamine cetyl sulfate, polyethylene glycol monostearate, ethylene glycol monostearate, polyoxyethylene hydrogenated castor oil, soybean lysophospholipid solution, polyoxyethylene sorbitan monooleate, and the like.

Preservatives include inorganic pigments such as phenoxyethanol, ethylparaben, butylparaben, zinc oxide, and the like.

Anti-inflammatory agents include glycyrrhizinic acid derivatives, salicylic acid derivatives, hinokitiol, zinc oxide, allantoin, and the like.
Whitening agents include placenta extract, glutathione, saxifraga extract, ascorbic acid derivatives, arbutin, and the like.

Blood circulation promoters include γ-oryzanol, sodium dextran sulfate, and the like.

Antiseborrheic agents include sulfur, thianthol, and the like.

Examples of thickening agents include carboxyvinyl polymers and the like.

pH adjusting agents include lactic acid, citric acid, malic acid, glycolic acid, sodium hydroxide, hydrotalcite, and the like.

(2) Effect as a Pharmaceutical for use with Wound Covering Materials

Zinc chloride as a pharmaceutical product of the zinc chloride hydroxide of the invention is free of a drying action or antimicrobial properties in contrast with zinc oxide and is useful as an active ingredient of pharmaceuticals. Also, it is useful as additives to other materials. Specifically, it can be used as an additive to a pharmaceutical composition as well as an additive to cosmetics and can exert different, synergistic, or enhanced effects on the substances to which the additives were added.

### EXAMPLES

The invention will be described in details using the examples below, but the invention is not limited thereto.

### Preparation Example 1

500 mL of 0.08 M aqueous ammonium chloride solution was prepared in a reaction vessel, and 1000 mL of 0.1 M aqueous zinc chloride solution was separately prepared as a drip reaction solution. 30 mass % aqueous sodium hydroxide solution was prepared as a pH adjusting solution.

Using a pH controller to which a pump was connected, the aqueous zinc chloride solution and aqueous sodium hydroxide solution were added dropwise with the above-mentioned aqueous ammonium chloride solution being maintained at pH 6.5 under stirring. After all of the aqueous zinc chloride solution was added, the reaction solution was stirred for 16 hours and was allowed to stand.

After that, the reaction solution was solid-liquid separated by centrifugation, and the obtained solid was washed with; centrifugation was repeated three times. The washed precipitate was vacuum dried to obtain dry powder of simonkolleite having the composition range shown in Formula (1).

### Preparation Example 2

pH at synthesis was set to 5.5 to 10, and various samples were prepared by the same process as in Preparation Example 1 while varying the pH at the time of preparation including that of Preparation Example 1 (pH 6.5). At pH 5.5, since the pH was too low, no precipitate was obtained.

### Dissolution Test

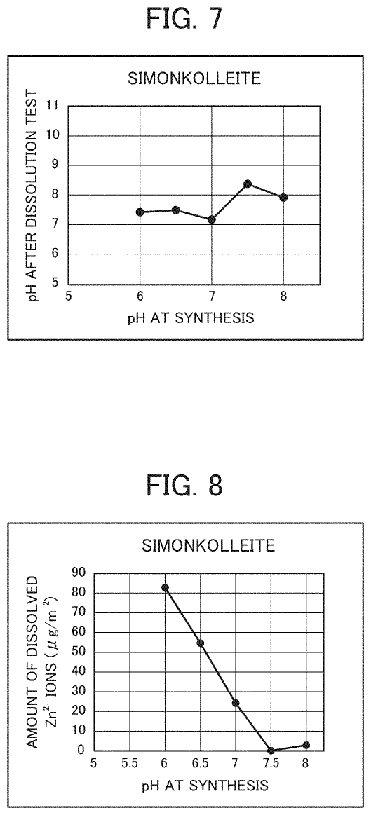

A dissolution test using 30 g of saline for 0.6 g of each dry powder obtained in Preparation Example 2 was performed by stirring method, and the amount of dissolved Zn²⁺ ions and pH were measured. In the method of the dissolution test, the BET specific surface area of the simonkolleite-containing zinc chloride hydroxide hydrate was adjusted to from 10 to 150 m²/g, the mass ratio of the simonkolleite-containing zinc chloride hydroxide hydrate to the saline was 1:50, and the time for stirring using a rotor at 500 rpm was set to be 3 hours. The pH and the amount of dissolved Zn²⁺ ions after the dissolution test were measured. The results are shown in FIGS. 7 and 8, and Table 1.

As shown in FIG. 8, the pH around 7.5 is considered to be a specific point in the synthesis of the simonkolleite-containing zinc chloride hydroxide hydrate, and an improved yield and crystallinity of simonkolleite can be observed at pH of less than 7.5. On the other hand, it is believed that a heterogenous phase occurs at pH of not less than 7.5, and zinc hydroxide is predominantly synthesized at pH of not less than 8.5.

TABLE 1

| | | | | | | |
|---|---|---|---|---|---|---|
| pH at synthesis | 5.5 | 6 | 6.5 | 7 | 7.5 | 8 |
| pH after dissolution | — | 7.42 | 7.49 | 7.17 | 8.37 | 7.91 |
| test | | | | | | |
| Amount of dissolved Zn²⁺ | — | 82.7 | 54.6 | 24.2 | 0.08 | 2.91 |
| ions after dissolution | | | | | | |
| test (μg/m²) | | | | | | |

### Pharmaceutical Example 1

To an approximately 500 g SD rat, 0.2 mL/500g of 2% Sederac (xylazine) was administered by intramuscular injection and the rat was sedated, and then, 2% of sevoflurane inhalation anesthetic was used to carry out a general anesthesia. Following local anesthesia by administering xylocaine (lidocaine+2% adrenaline) to the rat ventral side, a wound with all thickness skin loss of a diameter of 10 mm from epidermis reaching up to subcutaneous tissue was created, and 0.01 g of the powder of the above-mentioned Preparation Example 1 was applied to the wound created part wound with all thickness skin loss, and further covered with Duoactive which is a medical wound covering material.

### Tissue Staining and Hematoxylin-Eosin (H-E) Staining

The method of tissue staining was carried out as follows: wound site trimming (excision)→formalin fixation→degreasing treatment (immersed in xylene for 24 hours)→dehydration treatment.

The dehydration treatment was carried out as follows: samples were immersed in 70% ethanol for 12 hours and the ethanol was removed by volatilization, and then the samples were dehydrated by 80% ethanol, 90% ethanol, and 95.5% ethanol for 30 minutes each, and washed twice with xylene.

The samples were then embedded in paraffin→sections were prepared→H-E staining was carried out.

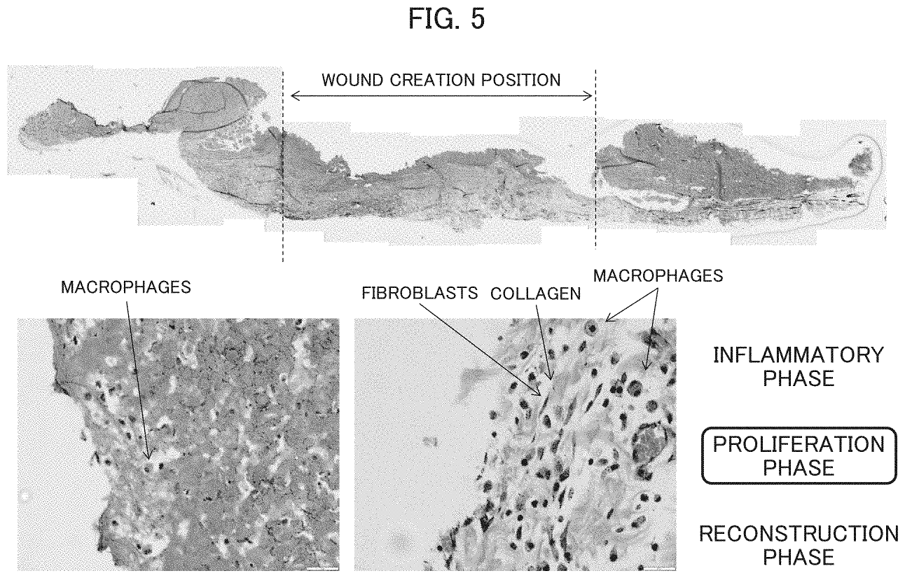

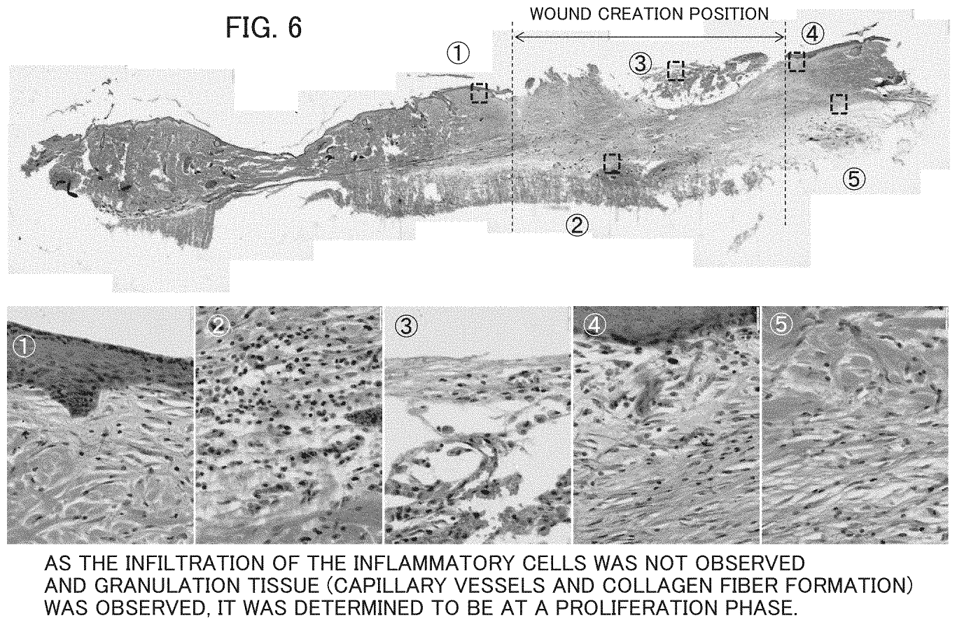

The cell nucleus was stained with hematoxylin in a blue-violet color and the cytoplasm, collagen fibers, and muscle fibers were stained in red color with eosin. →The sample after staining was enclosed in a prepared slide, and the microscopical observation results are shown in FIGS. 5 and 6.

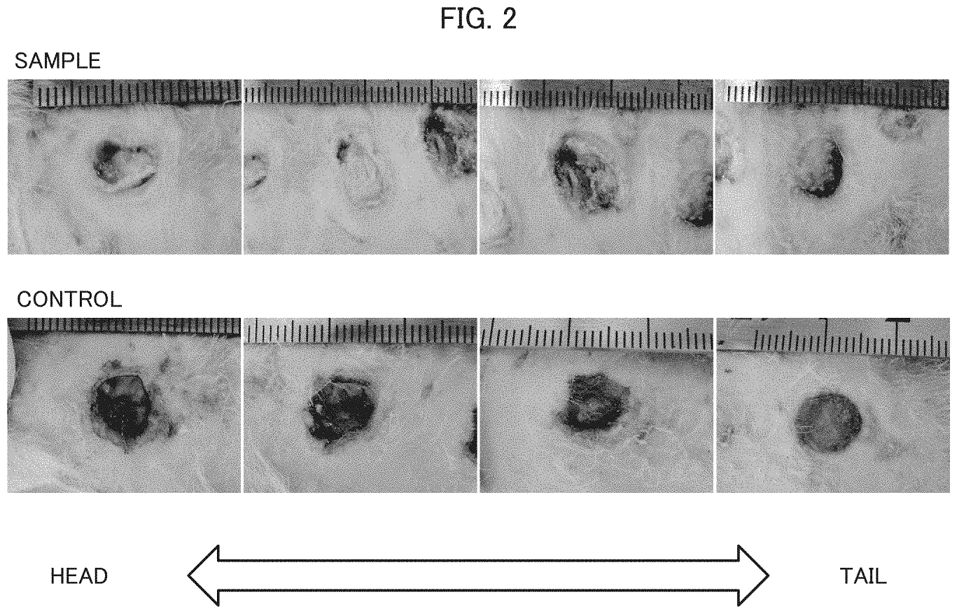

Observation results of the healing site after one week of covering with the wound covering material (FIG. 2): The therapeutic agent obtained in Preparation Example 1 was applied to the sample and then the sample was covered with the wound covering material. After one week, a white tissue was observed in the wound site of the sample; however, in a control which was only covered with Duoactive, a medical wound covering material, without using the therapeutic agent, the peritoneum was clearly seen, and the wound was not healed.

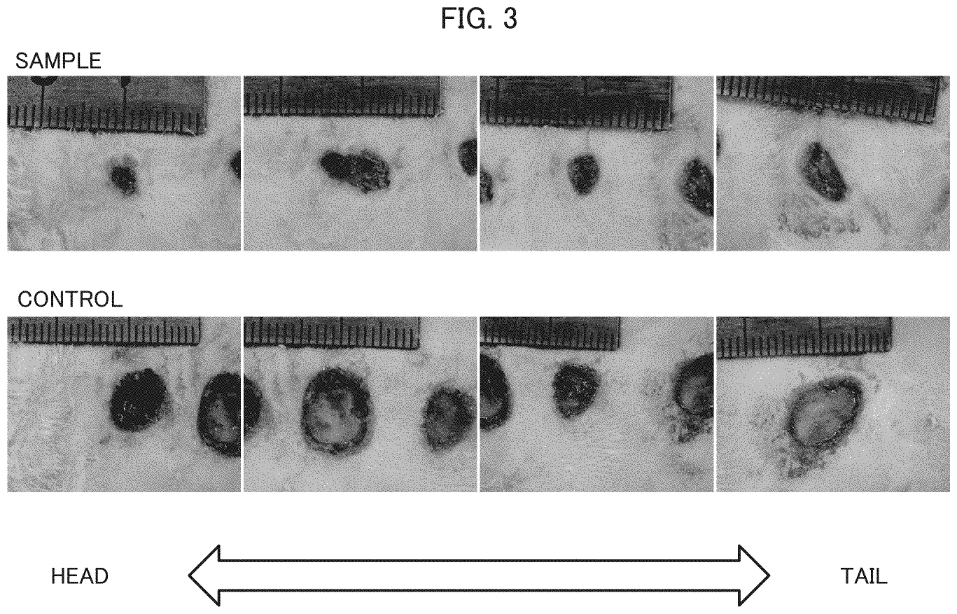

Observation results of the healing site after 2 weeks of covering with the wound covering material (FIG. 3): The therapeutic agent obtained in Preparation Example 1 was applied to the sample and then the sample was covered with the wound covering material. In the sample after 2 weeks, an obvious reduction of the wound site was observed and the peritoneum was not recognized. On the other hand, in the control where the therapeutic agent was not used, while a slight reduction in the wound site was observed, an unhealed site in which peritoneum is recognized was also observed.

### Measurement Method of Re-Epithelialization Rate

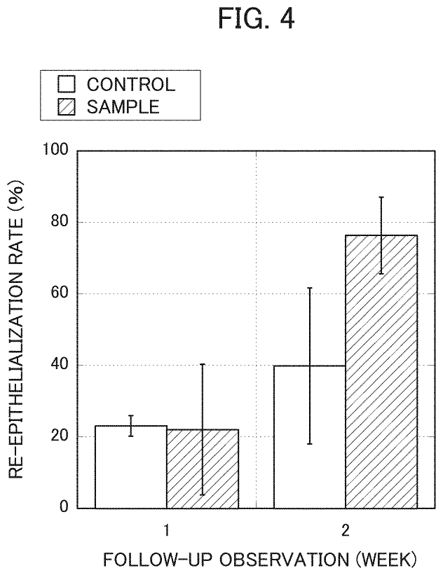

Magnified photographs of the skins were taken at the time of wound creation and after treatment. The initial wound site at the time of wound creation was measured and drawn into the photographs after 1 week and 2 weeks with a solid line. The area of the initial wound site W₀ was measured, the area of the unhealed site (Wt) after treatment was measured by Image J [open source published on the web; Wayne Rasband (NIH)], and the rate of re-epithelialization is calculated in % from the measured area using the following equation. The rates of re-epithelialization after treatment shown in FIG. 2 and FIG. 3 are shown in Table 2 and FIG. 4 together with those of the controls.

Re-epithelialization rate (%)=(W₀−Wₜ)/W₀×100%

TABLE 2

| Follow-up observation | Comparative | Examples of the |
|---|---|---|
| Time (week) | Examples | invention |
| 1 | 23.08 | 22.04 |
| 2 | 39.88 | 76.38 |

Based on the results shown in Table 2 and FIG. 4, the rate of re-epithelialization of the Pharmaceutical Example of the invention (sample) after 2 weeks was about 1.9 times that of the Comparative Example (control).

Re-epithelialization rate: a t-test was performed on data of two groups of Comparative Example and Pharmaceutical Examples of the invention after 2 weeks having different variances of re-epithelialization rate. The t-test was performed on three out of four wounds and no significant difference was observed due to a relatively large error bar; however, a significant difference was observed in the Pharmaceutical Examples of the invention in a case of calculation from the four wounds. In the calculation of the re-epithelialization rate, the white tissue of the control was evaluated as an unhealed region.

The calculation from the four wounds is shown in Table 3.

TABLE 3

| | Comparative | Examples of the |
|---|---|---|
| | Examples | invention |
| Number of examples | 4 | 4 |
| Average | 28.62 | 74.47 |
| Variance | 823.21 | 91.02 |
| Standard Deviation | 28.69 | 9.54 |
| Standard Error | 14.35 | 4.77 |

| | |
|---|---|
| Mean difference | −45.84 |
| Degree of freedom | 3 |
| t-value | −3.03 |
| p-value in t-test | 0.0435 |

FIGS. 5 and 6 are micrographs of skin cross sections showing histological observation results after 1 week and after 2 weeks. The top views of FIGS. 5 and 6 are overall views at a magnification of 36 times, and the bottom views of FIGS. 5 and 6 are enlarged views, at a magnification of 360 times, of the part shown in the top views.

FIG. 5 shows in its top view the healing state of the wound created part after one week and in its bottom view the enlarged view of the same. FIG. 6 shows in its top view wound created parts 2 and 3 and its bottom view micrographs showing histological assessments of the wound created part 2 and the wound created part 3, namely the wound created parts shown in the top view, after 2 weeks. Micrographs showing histological assessments of part 1, part 4, and part 5, other than the wound created parts, were shown together, and the progress of the wound healing was compared to the normal skin condition.

[Tissue observation results after 1 week (FIG. 5)]: When the therapeutic agent of the invention was applied, the presence of collagen, fibroblasts, or macrophages was confirmed, and therefore it was determined to be at a proliferation phase. Also, as the white tissue is believed to be a granulation tissue, if the white tissue is counted as a healed tissue in the calculation of the re-epithelization rate, the therapeutic agent of the invention could be determined to exhibit a very high skin regeneration ability.

[Tissue observation results after 2 weeks (FIG. 6)]: As the infiltration of inflammatory cells was not observed and granulation tissue (capillary vessels and collagen fiber formation) was observed, it was determined to be at a proliferation phase.

These results revealed that the therapeutic agent comprising simonkolleite of the invention is an effective wound treatment material. It is believed that skin regeneration or hair root regeneration can occur without causing inflammation. Accordingly, the therapeutic agent using the active ingredient of the invention has a therapeutic effect on a skin wound or skin roughness which does not reach up to dermis via epidermis, a therapeutic effect on a skin wound or skin roughness which reaches up to dermis via epidermis, or a therapeutic effect on a skin wound or skin roughness which is an all thickness skin loss reaching up to dermis via epidermis. Thus, the therapeutic agent using the active ingredient of the invention has not only a healing effect on the skin wound shown in the examples but also on similar wounds caused by severe roughness.

### Pharmaceutical Example 2

A wound with all thickness skin loss was created as in Example 1, and powder of sample from Preparation Example 2 was applied to each of the created wounds with all thickness skin loss and the wounds were further covered with Duoactive which is a medical wound covering material. The re-epithelialization rate was between 80 and 90%, and the therapeutic agent of the invention, which was obtained at pH in a range of not less than 6.0 and less than 7.5 as a manufacturing condition, has an excellent healing effect.

Table 4 below shows the pH after the dissolution test and the results of evaluation on wound healing effects after 2 weeks (re-epithelialization rate, collagen regeneration, hairball regeneration, and granulation formation).

TABLE 4

| | | | | | | | |
|---|---|---|---|---|---|---|---|
| pH at synthesis | 5.5 | 6 | 6.5 | 7 | 7.5 | 8 | — |
| pH after dissolution | — | 7.42 | 7.49 | 7.17 | 8.37 | 7.91 | — |
| test | | | | | | | |
| Amount of dissolved | — | 82.7 | 54.6 | 24.2 | 0.08 | 2.91 | — |
| Zn²⁺ ions after the | | | | | | | |
| dissolution test | | | | | | | |
| (μg/m²) | | | | | | | |

Healing state
Control
after 2 weeks

| | | | | | | | |
|---|---|---|---|---|---|---|---|
| Re-epithelialization | — | 1.9 | 1.9 | 1.8 | 1.4 | 1.3 | 1 |
| rate | | | | | | | |
| Collagen thickness | — | ⊚ | ⊚ | ⊚ | ○ | ○ | Δ |
| Collagen Orientation | | ⊚ | ⊚ | ⊚ | Δ | Δ | x |
| Hairballs | — | ⊚ | ⊚ | ⊚ | ○ | ○Δ | x |
| Granulation | | ⊚ | ⊚ | ○ | ○Δ | ○Δ | ○ |
| formation | | | | | | | |

The evaluation method of Table 4 is based on evaluation according to the above-described measurement method where the re-epithelialization rate of the control is regarded as 1. For the evaluation of collagen, thick collagen fibers extending in one direction were observed. With respect to hairballs, the observation results show whether the hairballs could be confirmed. For the evaluation of granulation formation, it was determined whether tissues composed of capillary vessels and fibroblasts were observed.

Collagen: For evaluation of collagen, collagen fiber thickness and orientation were assessed in comparison with healthy skin cells.

Collagen thickness: x: very thin

Δ: Thin

○: Slightly inferior

⊚: Same as healthy skin cells

### Collagen Orientation: x: Random

Δ: Very inferior

○: Slightly inferior

⊚: Same as healthy skin cells

Hairballs: x: No formation of hairballs is observed.

○Δ: Slight formation of hairballs.

○: Hairballs are formed.

⊚: The formation of hairballs is prominently observed.

Granulation formation: x: No granulation is observed.

○Δ: Slight granulation formation.

○: Granulation is observed.

⊚: Granulation is prominently observed.

The results, in which that of the control as a comparison is included, are shown in Table 4. Simonkolleite which is a pharmaceutical of the invention not only has a higher degree of re-epithelialization than the control, but also exhibits an equivalent recovery as a healthy skin tissue in the regenerated tissue, in particular, in the appearance of collagen, and thus it is found to have a great effect.

Accordingly, it is believed that the product of the invention may be applied to a commercially available organic based wound covering material, whereby the use of an inorganic based material of the active ingredient of the invention leads to hybridization of organic based/inorganic based materials, resulting in a great synergistic effect and a great improvement in the wound healing capability.

### Application Form

The pharmaceutical examples described above take an application form of powder for use in treatment techniques in which the basic zinc salt powder of the invention is spread onto the wound part and a medical wound covering material covers the wound part. The inventors conducted diligent research on the application form (treatment technique) of the basic zinc salt powder of the invention. The application form (treatment technique) and the healing effect when used as an ointment along with a covering material will be described hereinafter.

### Ointment Preparation Example 1

4.9 g of white vaseline was placed in a 50-mL beaker, heated to 60° C., and 0.1 g of liquid paraffin was added and mixed, after which 5 g of dry powder obtained in Preparation Example 1 was added thereto and sufficiently stirred to produce 10 g of ointment (amount of active ingredient: 50 mass %). The ointment was applied to the created wound with all thickness skin loss, and observation of wound appearance after 2 weeks and tissue observation of the wound part were carried out. The results are shown in Table 5.

### Comparative Example 1, using only an Ointment Base as a Control

Only the ointment base of white vaseline and paraffin which does not contain the active ingredient was applied as a comparative example to the wound with all thickness skin loss created as in the above-mentioned Ointment Preparation Example, and observation of wound appearance after 2 weeks and tissue observation of the wound part were carried out as a control with the base only. The results are shown in Table 5.

### Covering Material usage Examples 2 and 3

Using the powder obtained in Preparation Example 1, in Covering material usage example 2, 5 g of the dry powder of Preparation Example 1 was kneaded into 5 g of hydrogel (active ingredient: 50 mass %), and in Covering material usage example 3, the powder of Preparation Example 1 was fixed onto a hydrogel surface (wound contacting side) by a shot blasting method in an amount of 40 mass % (4 g of dry powder for 6 g of hydrogel), whereby Covering material usage example 2 and Covering material usage example 3 were prepared, respectively. Here, the hydrogels used are those used in the commercially available medical wound covering material Duoactive. Covering material usage example 2 and Covering material usage example 3 were each applied to the wound with all thickness skin loss as created in the above-mentioned Ointment Preparation Example, and observation of wound appearance after 2 weeks and tissue observation of the wound part were carried out. Results are shown in Table 5.

### Comparative Example 2, using only the Covering Material as a Control

A control (comparative example) in which the created wound with all thickness skin loss was merely covered with Duoactive was prepared, and observation of wound appearance after 2 weeks and tissue observation of the wound part were carried out. Results are shown in Table 5.

The Ointment Preparation Example 1, Covering material usage examples 2 and 3 were all evaluated for healing effect after 2 weeks (collagen thickness, collagen orientation, hairball regeneration, and granulation formation) and the results are shown. The evaluation criteria are the same as in Table 4.

The Simonkolleite which can be utilized as a pharmaceutical or medical device of the invention in Ointment Preparation Example 1 and Covering material usage examples 2 and 3 achieves recovery of the regenerated tissues to the same level as of a normal skin tissue, in particular, in terms of the appearance of collagen, and thus exhibits great effect, which is equivalent to the case the powder application in Pharmaceutical Example 1 and Pharmaceutical Example 2. The simonkolleite is also highly effective from the perspective of convenience at the clinical site.

TABLE 5

| | | Control | Covering | Covering | Control |
|---|---|---|---|---|---|
| | Ointment | only | material | material | only using |
| | Preparation | using | usage | usage | covering |
| | Example 1 | base | example 2 | example 3 | material |
| Collagen | ⊚ | Δ | ⊚ | ⊚ | Δ |
| thickness | | | | | |
| Collagen | ⊚ | X | ⊚ | ⊚ | X |
| Orientation | | | | | |
| Hairballs | ⊚ | X | ⊚ | ⊚ | X |
| Granulation | ⊚ | ◯Δ | ⊚ | ⊚ | ◯ |
| formation | | | | | |

### Evaluation of Test Results

The various test results described above can be summarized as described below. Since the wound healing agent causes an inflammatory response in wound healing when the pH value is 8 or greater, the pH of the wound healing agent is suitably in a range of neutral to 8. In view of the pH after the dissolution test in comparison to the pH at synthesis (FIGS. 7 and 8), the pH at synthesis exceeding an optimal pH is only 7.5, and the amount of dissolved Zn²⁺ ions that play an important role in the wound healing process is also small. This result shows that, when the pH is 7.5 at synthesis, Zn²⁺ ions do not bind to the active sites of MMPs, thus no destruction of the extracellular matrix occurs, and therefore cell migration in the wound healing process is not promoted, resulting in no promotion of wound healing. When the pH at synthesis is other than the region of around 7.5, it is within the optimal pH range, and as the amount of dissolved Zn²⁺ ions is large, the wound healing is considerably promoted. As shown in Table 4, the wound healing assessment on rats meets the above consideration. It is experimentally revealed that particularly the pH 7 at synthesis, at which the amount of dissolved zinc ions is large, is within the optimal pH range, and the wound healing is remarkable. As a reason for the dissolution behavior showing the specific behavior in a region around pH 7.5 at synthesis, it is assumed that a transient phase exists between a state where the yield of simonkolleite as a single phase simply increases until pH 7 and a state where a hydroxide phase such as Zn(OH)₂ other than simonkolleite is dominant at pH 8 or greater; however, the details are not clear at the moment.

### INDUSTRIAL APPLICABILITY

The zinc chloride hydroxide having excellent zinc ion sustained-release properties of the invention allows an appropriate sustained-release of zinc ions at a suitable condition when used as a pharmaceutical, and it can be used as an active ingredient of pharmaceuticals. The zinc chloride hydroxide of the invention as an active ingredient of pharmaceuticals has an excellent stability, and the manufacturing method thereof is simple; therefore, the zinc chloride hydroxide is industrially useful.

### <Specific Embodiments>

The invention includes the following as specific embodiments.

### [Claim 1]

An active ingredient of a zinc chloride hydroxide pharmaceutical having an excellent zinc ion sustained-release property, the active ingredient comprising at least one selected from the group consisting of zinc chloride, zinc hydroxide, and zinc oxide.

### [Claim 2]

The active ingredient of a zinc chloride hydroxide pharmaceutical having an excellent zinc ion sustained-release property according to claim 1, wherein the zinc chloride comprises at least one selected from the group consisting of zinc chloride, zinc chloride hydroxide, and zinc chloride hydroxide hydrate.

### [Claim 3]

An active ingredient of zinc chloride hydroxide pharmaceutical having an excellent zinc ion sustained-release property, wherein an amount of dissolved Zn²⁺ ions is from 0.25 to 100 μg/m² per unit surface area, and pH is not less than 7.0 and less than 8.3, after a dissolution test by stirring method performed on a simonkolleite-containing zinc chloride hydroxide hydrate;

where, in the dissolution test, a BET specific surface area of the simonkolleite-containing zinc chloride hydroxide hydrate is from 10 to 150 m²/g, a mass ratio of the simonkolleite-containing zinc chloride hydroxide hydrate to saline is 1:50, and a time for stirring at 37° C. using a rotor at 500 rpm is 3 hours.

### [Claim 4]

The active ingredient of zinc chloride hydroxide pharmaceutical having an excellent zinc ion sustained-release property according to claim 3, wherein a structure of Zn₅Cl₂(OH)₈·nH₂O is dominant in an XRD diffraction pattern of the simonkolleite-containing zinc chloride hydroxide hydrate, where a axis is from 6.3 to 6.345 and c axis is from 23.4 to 23.7.

### [Claim 5]

The active ingredient of zinc chloride hydroxide pharmaceutical having an excellent zinc ion sustained-release property according to claim 3 or 4, wherein the simonkolleite-containing zinc chloride hydroxide hydrate is represented by Formula (1) below, and a molar ratio of Zn to Cl is Zn/Cl=2.0−4.0;

Zn₄₋₆Cl₁₋₃(OH)₇₋₈·nH₂O (1)

where n is 0 to 6.

### [Claim 6]

The active ingredient of a zinc chloride hydroxide pharmaceutical having an excellent zinc ion sustained-release property according to claim 5, wherein the pharmaceutical is a therapeutic agent for a skin wound or skin roughness, and when the therapeutic agent is dissolved in saline and used, a ratio of the therapeutic agent to saline is from 0.1 g/L to 100 g/L, and the therapeutic agent is represented by Formula (1) above, and when n=0 (anhydrous), a zinc concentration relative to a total amount of the therapeutic agent is from 45 mass % to 75 mass % as metal zinc, and a zinc concentration in the saline aqueous solution of the therapeutic agent is from 0.045 g/L to 75 g/L.

### [Claim 7]

The active ingredient of a zinc chloride hydroxide pharmaceutical having an excellent zinc ion sustained-release property according to any one of claims 1 to 6, wherein the pharmaceutical is a therapeutic agent for a skin wound or skin roughness which reaches up to the dermis via the epidermis, or for pressure ulcers which reach up to peritoneum via epidermis and dermis.

### [Claim 8]

The active ingredient of a zinc chloride hydroxide pharmaceutical having an excellent zinc ion sustained-release property according to claim 6 or 7, wherein the therapeutic agent is in the form of powder, lotion, solution, cream, ointment, spray, or gel and is applied or sprayed to a part of a skin wound or skin roughness to be utilized.

### [Claim 9]

The active ingredient of a zinc chloride hydroxide pharmaceutical having an excellent zinc ion sustained-release property according to any one of claims 6 to 8, wherein the pharmaceutical is a medical device comprising a wound covering material for retaining the therapeutic agent and the skin wound or skin roughness in a closed environment.

### [Claim 10]

The active ingredient of a zinc chloride hydroxide pharmaceutical having an excellent zinc ion sustained-release property according to claim 9, wherein the wound covering material is at least one selected from the group consisting of polyurethane film dressing material, hydrocolloid dressing material, polyurethane foam dressing material, alginate covering material, hydrogel dressing material, hydropolymers, cellulose film, chitin wound covering material, and silk film.

### [Claim 11]

The active ingredient of a zinc chloride hydroxide pharmaceutical having an excellent zinc ion sustained-release property according to any one of claims 6 to 10, wherein the therapeutic agent for a skin wound or skin roughness is present by being applied to, contained in, or adhered to a wound covering material.

### [Claim 12]

The active ingredient of a zinc chloride hydroxide pharmaceutical having an excellent zinc ion sustained-release property according to any one of claims 9 to 11, wherein the wound covering material is made to carry the therapeutic agent when manufactured, or the therapeutic agent is fixed through spray, immersion, or application to an application side surface of the wound covering material that has been processed into a sheet form, whereby the therapeutic agent and the wound covering material are used as a medical device.

### [Claim 13]

The active ingredient of a zinc chloride hydroxide pharmaceutical having an excellent zinc ion sustained-release property according to any one of claims 1 to 8, wherein the pharmaceutical is a combined set of the therapeutic agent for a skin wound or skin roughness containing the zinc chloride hydroxide having an excellent zinc ion sustained-release property, and the wound covering material for retaining a skin wound or skin roughness in a closed environment.

### [Claim 14]

A method of manufacturing the active ingredient of a zinc chloride hydroxide pharmaceutical according to any one of claims 1 to 13, comprising maintaining an almost constant pH by acid or alkali in the presence of aqueous solution including chloride ions, continuously supplying zinc ions from an aqueous zinc solution, and precipitating zinc chloride hydroxide having a main component of simonkolleite.

### [Claim 15]

The method of manufacturing the active ingredient of a zinc chloride hydroxide pharmaceutical according to claim 14, wherein the pH is not less than 6.0 and less than 7.5, and a temperature is lower than 40° C.

### [Claim 16]

The method of manufacturing the active ingredient of a zinc chloride hydroxide pharmaceutical according to claim 14 or 15, wherein the zinc ion donor is at least one selected from the group consisting of zinc chloride and zinc nitrate.

### [Claim 17]

The method of manufacturing the active ingredient of a zinc chloride hydroxide pharmaceutical according to any one of claims 14 to 16, wherein the chloride ion donor is at least one selected from the group consisting of ammonium chloride and sodium chloride.

1. (canceled)

2. A zinc chloride hydroxide having an excellent zinc ion sustained-release property, which can be used as an active ingredient of a pharmaceutical, comprising zinc chloride hydroxide hydrate, wherein the zinc chloride hydroxide hydrate comprises simonkolleite.

3. The zinc chloride hydroxide having an excellent zinc ion sustained-release property according to claim 2, which can be used as an active ingredient of a pharmaceutical, wherein an amount of dissolved Zn²⁺ ions is from 0.25 to 100 μg/m² per unit surface area, and pH is not less than 7.0 and less than 8.3, after a dissolution test by stirring method performed on the simonkolleite-containing zinc chloride hydroxide hydrate;
where, in the dissolution test, a BET specific surface area of the simonkolleite-containing zinc chloride hydroxide hydrate is from 10 to 150 m²/g, a mass ratio of the simonkolleite-containing zinc chloride hydroxide hydrate to saline is 1:50, and a time for stirring at 37° C. using a rotor at 500 rpm is 3 hours.

4. The zinc chloride hydroxide having an excellent zinc ion sustained-release property according to claim 3, which can be used as an active ingredient of a pharmaceutical, wherein a structure of Zn₅Cl₂(OH)₈·nH₂O is dominant in an XRD diffraction pattern of the simonkolleite-containing zinc chloride hydroxide hydrate, where a axis is from 6.3 to 6.345 and c axis is from 23.4 to 23.7.

5. The zinc chloride hydroxide having an excellent zinc ion sustained-release property that can be used as an active ingredient of a pharmaceutical according to claim 3, wherein the simonkolleite-containing zinc chloride hydroxide hydrate is represented by Formula (1) below, and a molar ratio of Zn and Cl is Zn/Cl=2.0−4.0,
Zn₄₋₆Cl₁₋₃(OH)₇₋₈·nH₂O (1)
where n is 0 to 6.

6. The zinc chloride hydroxide having an excellent zinc ion sustained-release property according to claim 5, which can be used as an active ingredient of a pharmaceutical, wherein the pharmaceutical is a therapeutic agent for a skin wound or skin roughness, and when the therapeutic agent is dissolved in saline and used, a ratio of the therapeutic agent to saline is from 0.1 g/L to 100 g/L, and the therapeutic agent is represented by Formula (1) above, and when n=0 (anhydrous), a zinc concentration relative to a total amount of the therapeutic agent is from 45 mass % to 75 mass % as metal zinc, and a zinc concentration in the saline aqueous solution of the therapeutic agent is from 0.045 g/L to 75 g/L.

7. The zinc chloride hydroxide having an excellent zinc ion sustained-release property according to claim 2, which can be used as an active ingredient of a pharmaceutical, wherein the pharmaceutical is a therapeutic agent for a skin wound or skin roughness which reaches up to dermis via epidermis, or for pressure ulcers which reach up to peritoneum via epidermis and dermis.

8. The zinc chloride hydroxide having an excellent zinc ion sustained-release property according to claim 7, which can be used as an active ingredient of a pharmaceutical, wherein the therapeutic agent is in the form of powder, lotion, solution, cream, ointment, spray, or gel, and is applied or sprayed to a part of a skin wound or skin roughness to be utilized.

9. The zinc chloride hydroxide having an excellent zinc ion sustained-release property according to claim 7, which can be used as an active ingredient of a pharmaceutical, wherein the pharmaceutical is a medical device comprising a wound covering material for retaining the therapeutic agent and the skin wound or skin roughness in a closed environment.

10. The zinc chloride hydroxide having an excellent zinc ion sustained-release property according to claim 9, which can be used as an active ingredient of a pharmaceutical, wherein the wound covering material is at least one selected from the group consisting of polyurethane film dressing material, hydrocolloid dressing material, polyurethane foam dressing material, alginate covering material, hydrogel dressing material, hydropolymers, cellulose film, chitin wound covering material, and silk film.

11. The zinc chloride hydroxide having an excellent zinc ion sustained-release property according to claim 9, which can be used as an active ingredient of a pharmaceutical, wherein the therapeutic agent for a skin wound or skin roughness is present by being applied to, contained in, or adhered to the wound covering material.

12. The zinc chloride hydroxide having an excellent zinc ion sustained-release property according to claim 9, which can be used as an active ingredient of a pharmaceutical, wherein the wound covering material is made to carry the therapeutic agent when manufactured, or the therapeutic agent is fixed through spray, immersion, or application to an application side surface of the wound covering material that has been processed into a sheet form, whereby the therapeutic agent and the wound covering material are used as a medical device.

13. The zinc chloride hydroxide having an excellent zinc ion sustained-release property according to claim 7, which can be used as an active ingredient of a pharmaceutical, wherein the pharmaceutical is a combined set of the therapeutic agent for a skin wound or skin roughness containing the zinc chloride hydroxide having an excellent zinc ion sustained-release property, and the wound covering material for retaining a skin wound or skin roughness in a closed environment.

14. A method of manufacturing the zinc chloride hydroxide according to claim 2, comprising maintaining an almost constant pH by acid or alkali in the presence of aqueous solution including chloride ions, continuously supplying zinc ions from an aqueous zinc solution, and precipitating zinc chloride hydroxide having a main component of simonkolleite.

15. The method of manufacturing the active ingredient of a zinc chloride hydroxide pharmaceutical according to claim 14, wherein the pH is not less than 6.0 and less than 7.5, and a temperature is lower than 40° C.

16. The method of manufacturing the zinc chloride hydroxide according to claim 14, wherein the zinc ion donor is at least one selected from the group consisting of zinc chloride and zinc nitrate.

17. The method of manufacturing the zinc chloride hydroxide according to claim 14, wherein the chloride ion donor is at least one selected from the group consisting of ammonium chloride and sodium chloride.

18. The zinc chloride hydroxide having an excellent zinc ion sustained-release property that can be used as an active ingredient of a pharmaceutical according to claim 4, wherein the simonkolleite-containing zinc chloride hydroxide hydrate is represented by Formula (1) below, and a molar ratio of Zn and Cl is Zn/Cl=2.0−4.0,
Zn₄₋₆Cl₁₋₃(OH)₇₋₈·nH₂O (1)
where n is 0 to 6.

19. The zinc chloride hydroxide having an excellent zinc ion sustained-release property according to claim 3, which can be used as an active ingredient of a pharmaceutical, wherein the pharmaceutical is a therapeutic agent for a skin wound or skin roughness which reaches up to dermis via epidermis, or for pressure ulcers which reach up to peritoneum via epidermis and dermis.

20. The zinc chloride hydroxide having an excellent zinc ion sustained-release property according to claim 4, which can be used as an active ingredient of a pharmaceutical, wherein the pharmaceutical is a therapeutic agent for a skin wound or skin roughness which reaches up to dermis via epidermis, or for pressure ulcers which reach up to peritoneum via epidermis and dermis.
